(19) United States
(12) Patent Application Publication
(10) Pub. No.: US 2021/0059535 A1
(43) Pub. Date: Mar. 4, 2021

(54) HANDHELD ULTRASOUND TRANSDUCER ARRAY FOR 3D TRANSCRANIAL AND TRANSTHORACIC ULTRASOUND AND ACOUSTOELECTRIC IMAGING AND RELATED MODALITIES

(71) Applicant: ARIZONA BOARD OF REGENTS ON BEHALF OF THE UNIVERSITY OF ARIZONA, TUCSON, AZ (US)

(72) Inventors: Russell S. Witte, Tucson, AZ (US); Yexian Qin, Tucson, AZ (US); Matthew O'Donnell, Seattle, WA (US); Zhen Xu, Ann Arbor, MI (US); Charles Ingram, Tucson, AZ (US)

(21) Appl. No.: 16/644,755

(22) PCT Filed: Sep. 7, 2018
(86) PCT No.: PCT/US2018/049938

§ 371 Date: Mar. 5, 2020

Related U.S. Application Data
(60) Provisional application No. 62/555,437, filed on Sep. 7, 2017.

(51) Int. Cl. A61B 5/00; A61B 8/00; A61B 8/08; A61B 8/06; G01S 15/89; G01S 7/52

(52) U.S. Cl. CPC A61B 5/0093; A61B 8/4488; A61B 8/4461; A61B 8/483; A61B 8/0816; G01S 7/52079; A61B 8/488; A61B 8/5246; A61B 8/0883; G01S 15/8993; G01S 15/8925; A61B 8/06

(57) ABSTRACT

A two-dimensional wideband ultrasound transducer array for three or four-dimensional (volume+time) non-invasively imaging/mapping of electrical current in, for example, the brain through the skull, or the heart. The probe also has unique capabilities for three-dimensional transcranial or transthoracic pulse echo ultrasound (tissue structure, motion, bone thickness) and doppler blood flow imaging. The handheld device interfaces with an ultrasound delivery system for applications to human brain or heart imaging, ultrasound neuromodulation, and therapy. The handheld ultrasound array enables three-dimensional steering of an ultrasound beam through the human skull or chest for ultrasound, doppler, and acoustoelectric imaging and related modalities to aid in the diagnosis and treatment of brain or heart disorders.

### CROSS REFERENCE TO RELATED APPLICATION(S)

This application claims priority to U.S. provisional patent application No. 62/555,437, filed on Sep. 7, 2017, which is hereby incorporated herein by reference in its entirety.

### GOVERNMENT SPONSORSHIP

This invention was made with government support under Grant No. R24 MH109060 awarded by NIH. The government has certain rights in the invention.

### FIELD OF THE INVENTION

Embodiments are in the field of systems and methods for imaging body parts. More particularly, embodiments disclosed herein relate to systems and methods for 3D or 4D non-invasive imaging of body parts.

### BACKGROUND OF THE INVENTION

In the brain, human behavior and function evolve at the millisecond and millimeter scales. However, despite the proliferation of brain imaging modalities over the last 50 years, there remains an unmet need for a technique capable of noninvasive, high resolution electrical mapping of the human brain. Cutting-edge electroencephalography (EEG) and magnetoencephalography (MEG) suffer from poor spatial resolution and inaccurate mapping due to the ambiguous spread of the electric or magnetic fields through the head. In other words, there is no unique solution to the “inverse problem.” Other common modalities, such as functional magnetic resonance imaging (fMRI) and near infrared spectroscopy (fNIR), have relatively poor spatial resolution (˜1 cm) and typically capture slowly-evolving “intrinsic” neural activity that is loosely related to the underlying electrical activity.

Thus, it is desirable to provide embodiments of a system and method for 3D or 4D non-invasive imaging that do not suffer from the above drawbacks.

These and other advantages of the present invention will become more fully apparent from the detailed description of the invention herein below.

### SUMMARY OF THE INVENTION

Embodiments are directed to a method for 3D or 4D non-invasive imaging. In an embodiment, the method comprises: providing a 2D wideband ultrasound transducer array; delivering an ultrasound beam non-invasively to a body part using the transducer array, the ultrasound beam being in the form of plane waves with no focus, spherically focused waves, or cylindrically focused waves; and mapping electrical current in the body part using the providing and delivering steps. Embodiments of the method are capable of 3D or 4D mapping of electrical current in, for example, the brain through the skull, or the heart.

Embodiments are also directed to a 3D or 4D non-invasive imaging system. In an embodiment, the non-invasive imaging system comprises: a 2D wideband ultrasound transducer array that delivers an ultrasound beam non-invasively to a body part, the ultrasound beam being in the form of plane waves with no focus, spherically focused waves, or cylindrically focused waves; and a mapping system that maps electrical current in the body part using information obtained via the ultrasound beam delivered by the transducer array. Embodiments of the system are capable of 3D or 4D mapping of electrical current in, for example, the brain through the skull, or the heart.

Additional embodiments and additional features of embodiments for the method for 3D or 4D non-invasive imaging and 3D or 4D non-invasive imaging system are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It's understood, however, that the inventive concepts disclosed herein are not limited to the precise arrangements and instrumentalities shown in the figures. The detailed description will refer to the following drawings in which like numerals, where present, refer to like items.

FIGS. 1A and 1B are diagrams illustrating plots of a one-way impulse response of an ultrasound transducer of a simulation (FIG. 1A) and of an experimental measurement (FIG. 1B). The central frequency of the transducer is 0.5 MHz and bandwidth of 90%.

FIG. 2 is a diagram illustrating plots of geometry and setup for the present model using a focused single element transducer. Current is simulated as a cylinder with different diameters (D).

FIGS. 3A and 3B are diagrams illustrating plots of examples of coded ultrasound excitation for acoustoelectric (AE) imaging. FIG. 3A shows traditional linear chirp (f₀=0.05 MHz and f₁=0.95 MHz). FIG. 3B shows engineered amplitude modulated (ramp) chirp (f₀=0.21 MHz and f₁=1.2 MHz).

FIGS. 4A and 4B are diagrams illustrating plots of a spectrum of coded ultrasound excitation for AE imaging. FIG. 4A shows traditional linear chirp. FIG. 4B shows engineered amplitude modulated (ramp) chirp. The upper right curve is the simulated impulse response function.

FIG. 5 is a diagram illustrating a target signal and compressed output signal generated by minimizing the cost function to fit the target signal. The least square error is less than 0.7.

FIG. 6 is a diagram illustrating an envelope of an AE signal generated by traditional linear chirp (solid thick line) and ramp chirp (solid thin line) with different diameter current sources. The dashed lines indicate the bottom edge, center, and top edge of the current source cross section, respectively. Each signal was normalized by the peak pressure produced by the transducer.

FIG. 7 is a diagram illustrating cross sectional B-mode images of AE signal generated by traditional linear chirp (left) and ramp chirp (right) with different diameters (D) of the current source.

FIG. 8 is a diagram illustrating a magnitude of the AE signal with different diameters of the current source for the (a) top edge, (b) center, and (c) bottom of the current source. The signal is larger in the center for the nonlinear chirp for diameters >3 mm.

FIG. 9 is a diagram illustrating an experimental schematic for acoustoelectric brain imaging (ABI) using a 2D ultrasound array. An adult human skull cap was immersed in 0.9% saline with an acoustic window made of Mylar®. The ultrasound array was in contact with an acoustic window coupled with a layer of ultrasonic gel. Platinum electrodes were inserted in saline above the skull cap for injecting arbitrary current waveforms. A custom-made signal conditioning system was used to separate and amplify the low frequency (LF) waveform (3 cycles at 200 Hz) and high frequency AE signals (HF). These signals were then amplified and digitized by a data acquisition (DAQ) system.

FIG. 10 is a diagram illustrating a plot of pressure amplitude with and without a human adult skull cap. The plot was made with an acoustic pressure calibration for 2D ultrasound array (H235) using the Onda® hydrophone (HGL-0200) and a pre-amplifier AG2010. The ultrasound array was driven by a Verasonics ultrasound system with a short pulse at 20V. The hydrophone was placed 35 mm above the ultrasound array at the elevational geometrical focus. The measured pressure was recorded by the NI DAQ PXI-5105 at a 20 MHz sampling rate.

FIG. 11 is a diagram illustrating a plot of a comparison of AE signals with and without the human skull cap in the ultrasound propagation path. The high amplitude signal is the AE signal acquired without the skull cap. The lower amplitude signal was acquired when a skull cap was put on top of the acoustic window between the ultrasound array and dipole.

FIGS. 12A and 12B are diagrams illustrating a B-Mode image of transcranial Acoustoelectric Brain Imaging (tABI) with a dipole current source (3-cycle, 200 Hz) separated by 10 mm at t=9 ms (FIG. 12A). The dynamic range is 15 dB; FIG. 12B is a plot showing injected current into the medium measured across a 1 Ohm resistor.

FIG. 13 is a diagram illustrating a plot of an initial sensitivity measurement of tABI using the custom H235 2D ultrasound array and current source configuration from the previous section consisting of two platinum electrodes separated by 10 mm.

FIG. 14 is a diagram illustrating a plot of a baseline SNR estimate at different current levels with and without skull cap inserted.

FIG. 15 is a diagram illustrating geometry of a transcranial transducer array.

FIG. 16 is a diagram illustrating a comparison chart of parameters for various transcranial transducer arrays.

FIG. 17A is a diagram illustrating a 44×3 curved strip transcranial transducer array.

FIG. 17B shows pressure at beam steered focuses. To compare pressure at desired focus and natural focus, acoustic beam was steered and resultant focal pressure was mapped to each position in x-y plane (z=5 cm).

FIG. 18A is a diagram illustrating a 44×3 flat strip transcranial transducer array. FIG. 18B shows pressure at beam steered focuses. To compare pressure at desired focus and natural focus, acoustic beam was steered and resultant focal pressure was mapped to each position in x-y plane (z=5 cm).

FIG. 19A is a diagram illustrating a 18×7 curved strip transcranial transducer array. FIG. 19B shows pressure at beam steered focuses. To compare pressure at desired focus and natural focus, acoustic beam was steered and resultant focal pressure was mapped to each position in x-y plane (z=5 cm).

FIG. 20A is a diagram illustrating a 18×7 flat strip transcranial transducer array. FIG. 20B shows pressure at beam steered focuses. To compare pressure at desired focus and natural focus, acoustic beam was steered and resultant focal pressure was mapped to each position in x-y plane (z=5 cm).

FIG. 21 is a diagram illustrating a chart of parameters for a transthoracic transducer array.

FIG. 22A is a diagram illustrating a 18×7 curved strip transthoracic transducer array. FIG. 22B shows a plot of pressure vs. elevation (depicting pressure at beam steered focuses).

FIGS. 23A-23C are diagrams illustrating electrical impedance (FIG. 23A), received excitation response (FIG. 23B), and electrical input impedance (FIG. 23C), all vs. frequency.

FIG. 24 is a flowchart illustrating an embodiment of a method for 3D or 4D non-invasive imaging, in accordance with an embodiment.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical system that images body parts and typical method for imaging body parts. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

There are potentially many different target areas/applications for the method/system described in this disclosure, although the brain is the focus herein for purposes of explanation only. Application to the heart is also briefly discussed.

1. Improving Sensitivity in Acoustoelectric Imaging with Coded Excitation and Optimized Inverse Filter

Acoustoelectric imaging (AEI) is based on the interaction between a pressure wave and tissue resistivity to map electrical current at high spatial resolution. This approach overcomes limitations with conventional bioelectrical imaging, which typically suffers from poor resolution due to the ambiguous conductivity distribution between the current sources and detection electrodes. The inventors have shown in a variety of applications, including the live rabbit heart, the magnitude of the AE signal at physiological current is weak (˜1 μV). In this disclosure, the inventors examine the role of the pulse waveform in amplifying the AE signal and improving the signal-to-noise ratio for imaging. Using both simulation and bench-top experiment with a standard broadband ultrasound transducer, the inventors analyze the effects of nonlinear coded excitation with optimized compression. Compared to a short linear frequency modulated pulse (chirp), the nonlinear chirp with optimized inverse filtering can improve the signal to noise ratio (SNR) under certain conditions by >6 dB while preserving high spatial resolution.

1.1. Theory

Medical procedures, such as cardiac ablation therapy, require precise and accurate electrical mapping of tissue as feedback during treatment. However, conventional mapping methods are time consuming, require an array of invasive electrodes (e.g., intracardiac recording) or exhibit low spatial resolution (e.g., surface electrocardiography). AE imaging overcomes this limitation by localizing an electrical measurement to the focus of an ultrasound beam. The principal of AE imaging is based on the AE effect, the modulation of electric resistivity induced by a pressure wave. The induced AE potential V^{AE} due to a propagating pressure wave in a conductive medium with current field is expressed as

$${V}^{AE} ( {x}_{1} , {y}_{1} , t ) = - {K}_{ρ} {P}_{0} \int \int \int ( {J}^{L} \cdot {J}^{I} ) b ( x - {x}_{1} , y - {y}_{1} , z ) \times a ( t - \frac{z}{c} ) dxdydz , \qquad ( 1 )$$

where K is the interaction constant, ρ is the resistivity of the medium, P₀ is the pulse pressure amplitude. J^{L} is the lead field, b(x,y,z) is the beam pattern of the ultrasonic wave, c is the speed of sound, and a(t−z/c) is the ultrasound pulse waveform propagating over time t. When a burst of ultrasound pulses is steered within a volume, this equation describes how maps of current densities (direction and amplitude) in tissue can be obtained with AEI. The inventors have demonstrated 4D AEI in a variety of applications, such as the live rabbit heart, peripheral nerve bundle, and human head/brain phantom. From equation (1), the sensitivity and resolution of the AE signal is determined by the ultrasound beam pattern, bandwidth, pressure amplitude, and pulse waveform. This disclosure examines the effect of the pulse waveform using a standard ultrasound transducer on the sensitivity and resolution of AEI. By improving the time bandwidth product, coded ultrasound excitation pulses, such as linear chirps, are widely used in radar transmission and signal processing. They have also been used to improve SNR in acoustoelectric imaging studies, including the live rabbit heart. A popular coded excitation signal for ultrasound imaging is the linear chirp expressed as

$$s ( t ) = A ( t ) sin ( {ϕ}_{0} + 2 π ( {f}_{0} t + β \frac{{t}^{2}}{2} ) ) , \qquad ( 2 )$$

where A (t) is the apodization, φ₀ is the starting phase, f₀ is the starting frequency, and β is the rate of frequency change (or sweep rate) defined as

$$β = \frac{{f}_{1} - {f}_{0}}{T} , \qquad ( 3 )$$

where f₁ is the final frequency and T is the duration of the chirp. When a coded excitation signal is used, the signal to noise ratio (SNR) can be raised by more than 15 dB with effective penetration depth. Resolution and penetration depth are both important in ultrasound imaging. However, there is typically a tradeoff between resolution and penetration in most cases. Pulse compression techniques developed for radar systems have been used to mitigate this limitation. They employ a long pulse for higher radiated energy to improve the range resolution compared to a short pulse. With frequency encoding and optimal compression, the longer pulse can improve SNR while preserving the spatial resolution of a short pulse.

The most popular filter used for pulse compression is the matched filter. The filter coefficients for the matched filter (f_{match}) are usually the same as the original coded excitation signal with reverse order in time and represented by

f_{match}(t)=s(−t) (4)

and with an impulse response function, h(t), of the transducer, the AE signal (S_{AE}) after compression can be expressed as

S_{AE,compressed}(t)=(s(t)⊗h(t)⊗f_{match}(t), (5)

where ⊗ is the convolution operator. Therefore, for a given impulse response of the transducer and current field, the SNR of the AE signal can be improved by using an optimally designed excitation waveform and compression filter. Due to the volume integration in Eq. 1, it is desirable to generate unbalanced or “unipolar” pulses to improve sensitivity of AE imaging in uniform current fields. However, standard ultrasound transducers produce balanced pulses with similar positive and negative excursions. In this disclosure, the inventors designed a coded excitation signal and an inverse filter based on a standard ultrasound transducer with limited bandwidth to produce a “quasi-unipolar” pulse with the goal of amplifying the AE signal under certain conditions.

1.2. Methods

A. Acoustoelectric Simulations

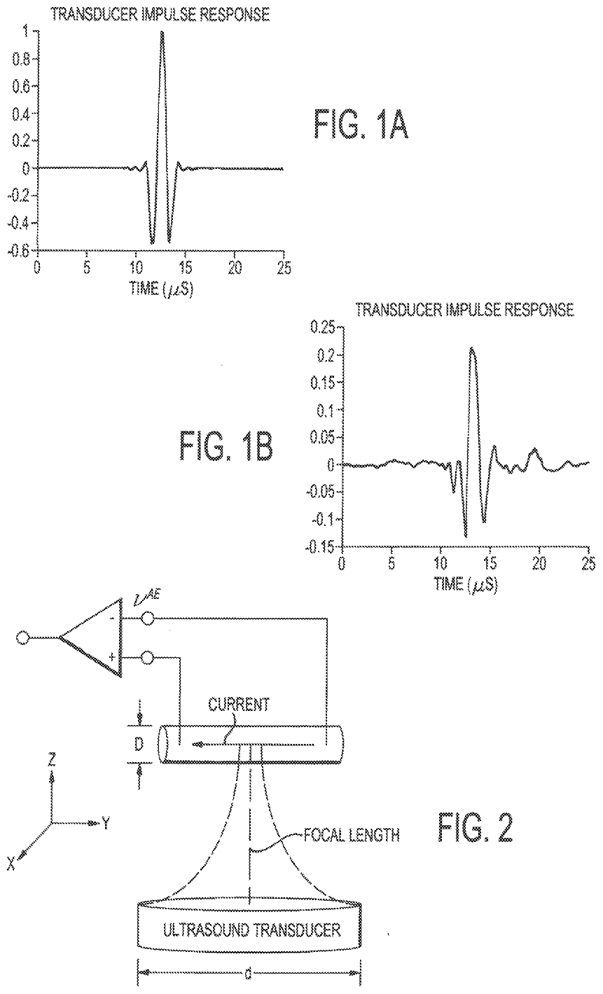

The ultrasound transducer and pressure field were simulated in FOCUS™. The transducer was modeled as a single concave element with a focal length of 2.15 inches and a diameter (D) of 1.5 inches, which matched a commercially available transducer (Olympus NDT, V389, 0.5 MHz). The one-way impulse response was modeled as a Gaussian pulse with 90% bandwidth (FIG. 1A). The experimentally measured impulse response (determined from pulse echo) was also used in the simulation (FIG. 1B). The shape of the current field was confined to a long cylinder with different diameters relative to the acoustic wavelength (FIG. 2).

B. Coded Excitation

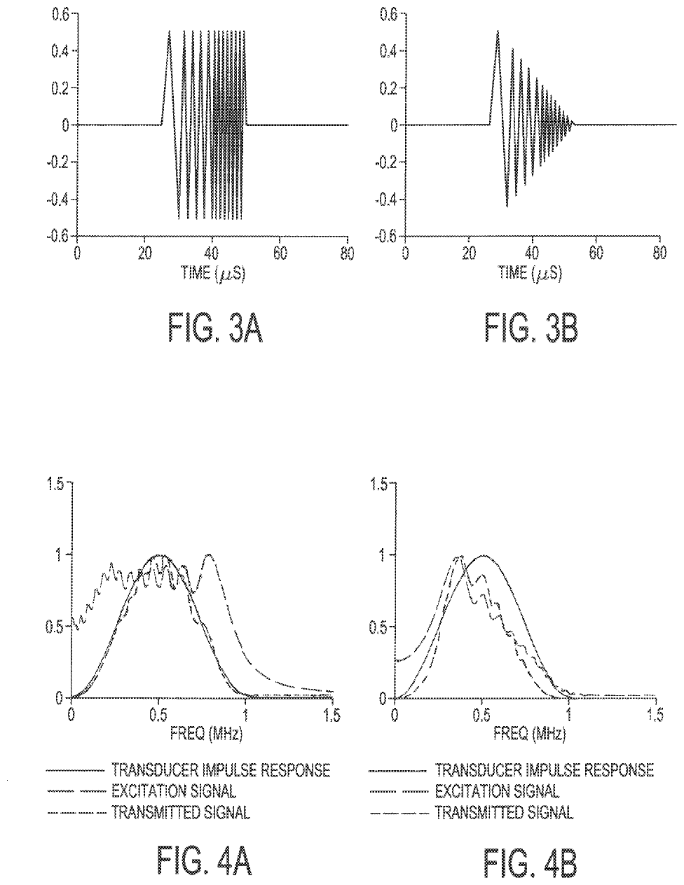

The duration of the designed linear and nonlinear chirps is 25 μsec. The linear chirp has an f₀ and f₁ of 0.05 MHz and 0.95 MHz, respectively, and the nonlinear chirp has an f₀ and f₁ of 0.21 MHz and 1.2 MHz, respectively. Whereas the apodization for the linear chirp is rectangular, the apodization for the nonlinear chirp was a negative ramp, producing more weight towards the low frequency spectrum. The time waveforms are displayed in FIGS. 3A and 3B, whereas their spectra appear in FIGS. 4A and 4B. Each chirp was used to produce AE signals and images with the same electrical stimulation conditions.

C. Filter Design

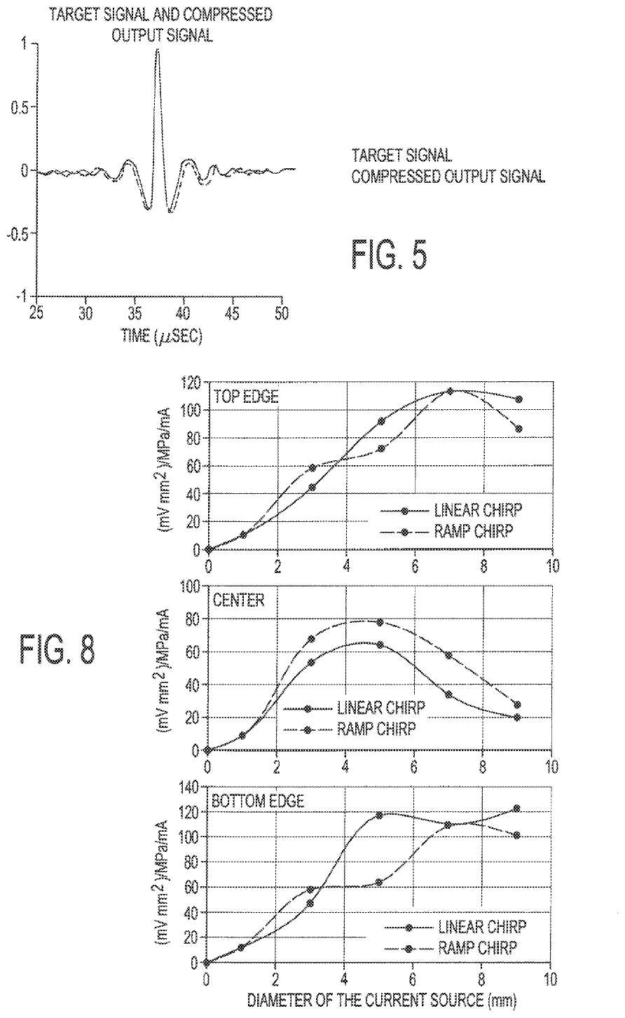

The ideal desired shape of the AE signal is unipolar. However, because a transducer has limited bandwidth and the signal integrates to 0, an ideal unipolar is not feasible. A quasi-unipolar shape is possible within the spectrum of the transducer, which approaches the unipolar pulse. A finite impulse response (FIR) filter was designed to compress the AE signal to the target waveform according to Eq. (5). The desired/target quasi-unipolar AE pulse is displayed in FIG. 5 (dashed curve). The AE signal can be expressed as

S_{AE}(t)=(sᵣₐₘₚ(t)⊗h(t))⊗fᵢₙᵥₑᵣₛₑ(t) (6)

where sᵣₐₘₚ(t) is the designed chirp with a negative ramp window mentioned in the previous section and fᵢₙᵥₑᵣₛₑ is the designed FIR to compress and shape the pulse. The filter coefficients were guided by the target signal using a least-squares minimization procedure expressed as

Σᵢ₌₁^{N}(s_{target,i}−S_{AE,i})², (7)

where N is the duration of the signal. The S_{AE}(t) using the simulated impulse response of the 0.5 MHZ transducer is displayed in FIG. 5 along with the target waveform.

1.3. Results

To investigate how different pulse waveforms might influence the AE signal, the transmitted pressure field has been normalized by the root mean square (RMS) before the integration of Eq. 1. The injected current J^{l} was

$$\frac{1 mA}{{cm}^{2}}$$

and parallel to the current field J^{L} for simplification. The inventors examined the effect of quasi-unipolar pulses for AE imaging of cylindrical current sources with varying diameters from 0.1 to 9 mm. The inventors analyzed the AE signal amplitude for linear and nonlinear chirps at three locations along the circular cross section: top edge, center, and bottom of the current source.

A. AE Signal for Linear and Nonlinear Chirps

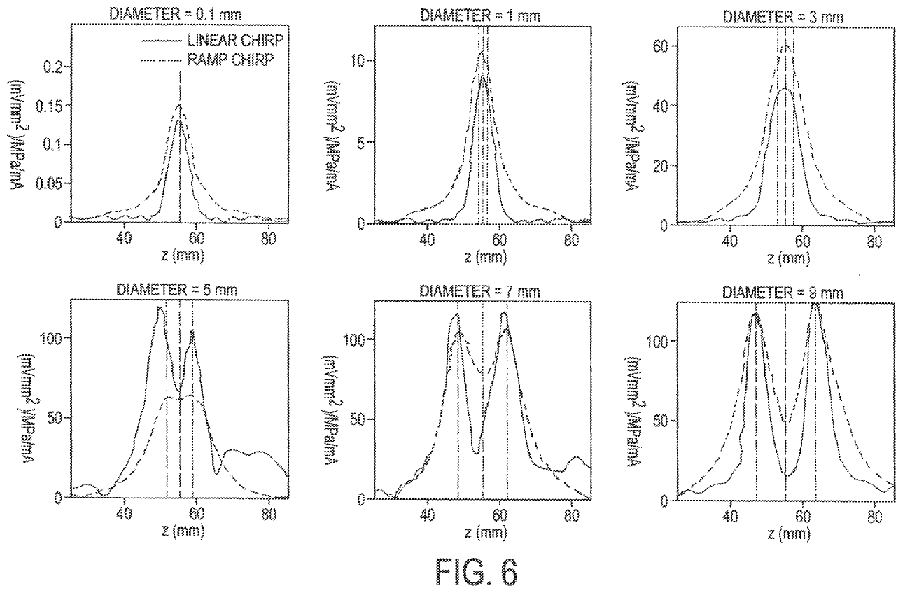

The A-line (envelope) of the AE signal generated by the linear and nonlinear chirps are depicted in FIG. 6. For both cases, the compression filter improves resolution and SNR and the edges of the cylinder produce the strongest AE signal. However, using the nonlinear chirp, a comparatively larger signal is generated inside the cross section for diameters of the current source >3 mm (i.e., greater than the acoustic wavelength of 3 mm).

B. Cross-sectional AE Images

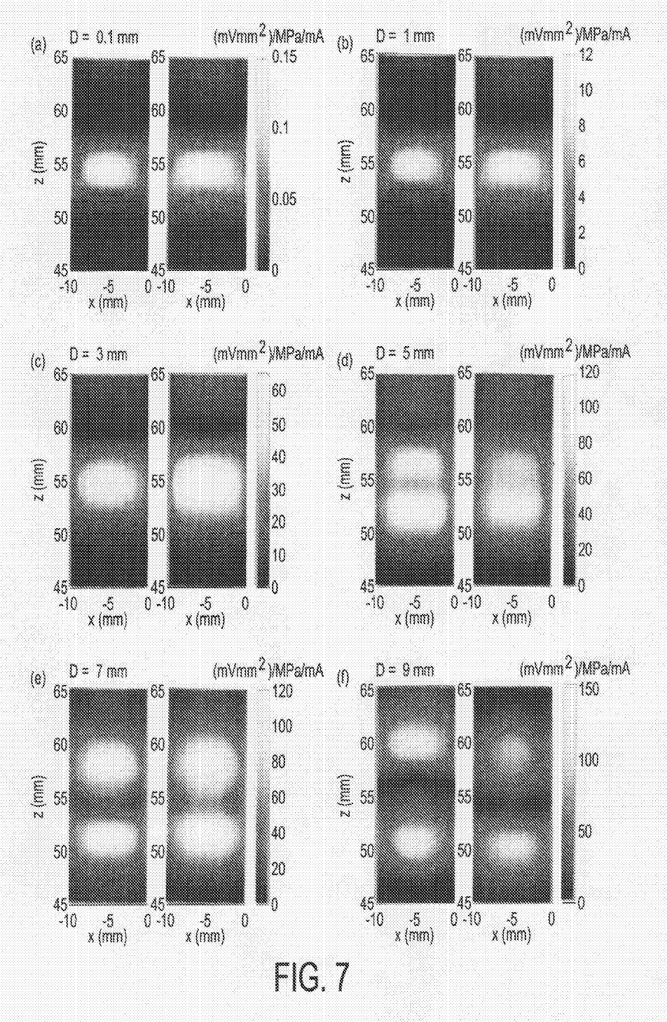

Cross-sectional B-mode AE images for the linear and nonlinear chirp excitations using experimentally measured transducer impulse response are displayed in FIG. 7 Similar to the results with the Gaussian impulse response, the decrease in signal at the interior of the current source with the linear chirp after compression is evident. The negative ramp modulated chirp, on the other hand, preserves much more of the signal inside the current source as the diameter increases. A comparison of the magnitude at three different locations—top edge, center, and bottom edge—are plotted in FIG. 8.

1.4. Discussion and Conclusion

Compared to traditional linear chirps with matched filter, the inventors have demonstrated that nonlinear chirps with low frequency weighting combined with an optimal inverse filter improves the sensitivity of the AE signal in regions of uniform current and away from sharp current gradients. Unlike traditional approaches, our method exploits the lower frequencies to generate a stronger signal in regions of near uniform current densities and reduce cancellation caused by a balanced ultrasound pulse. The quasi-unipolar signal, therefore, can enhance the magnitude of the central region of the current source. The approach may be combined with other methods of excitation for AE imaging for biomedical applications, including ultrafast plane wave imaging.

The approach using nonlinear coded waveforms with inverse filter is not limited to any specific transducer. Thus, a custom designed “unipolar” transducer with low frequency weighting is not required to achieve the effect described in this disclosure. It should be noted that there is a tradeoff between the efficiency and shape of the quasi-unipolar pulse. Since the nonlinear chirp has more weight at the lower frequency part of the transducer band, the transmit signal is less efficient. However, this can usually be compensated by increasing the amplitude of the drive signal to correct for a loss in efficiency.

Thus, AE imaging with quasi-unipolar pulses may be an important strategy for amplifying the weak AE signal observed in a physiologic setting. Experiments are underway to confirm the modeling results and further optimize the design of the coded excitation and inverse filter.

### 2. Performance of a Transcranial Ultrasound Array Designed for 4D Acoustoelectric Brain Imaging in Humans

Noninvasive electrical brain imaging in humans suffers from poor spatial resolution due to the uncertain spread of electric fields through the head. To overcome this limitation, the inventors employed 4D tABI based on the acoustoelectric effect for mapping current densities at a spatial resolution confined to the ultrasound focus. AE imaging exploits an interaction between a pressure wave and tissue resistivity, which was demonstrated for mapping the cardiac activation wave in the rabbit heart. The inventors have extended this modality for mapping the human brain noninvasively. This disclosure describes the performance of a 2D ultrasound array designed for tABI in humans. The performance of, for example, a custom 0.6 MHz 2D ultrasound array designed for tABI through the adult human skull. Time-varying current was injected between two electrodes in 0.9% saline to produce a dipole at well-controlled current densities. A distant recording electrode was placed in the saline bath to detect the AE signal as the ultrasound beam was electronically steered in 3D near the dipole. At each beam position, a burst of ultrasound pulses was delivered to reconstruct the time-varying current. The AE amplitude was measured with and without an adult human skull and at different current amplitudes. The AE signal could be detected at depths greater than 40 mm from the surface of the skull. Sensitivity for detecting the AE signal through bone was 1.47 μV/(MPa*mA/cm²). The noise equivalent current densities normalized to 1 MPa were 1.3 and 1.8 mA/cm² with and without the skull, respectively. Further optimization of ABI instrumentation and beamforming may be contemplated to push the detection limit towards small neural currents through thick skull and will lead to a new noninvasive modality for real-time electrical brain imaging in humans.

2.1. Theory

The inventors employed tABI as a potentially revolutionary modality for real-time, high resolution electrical brain mapping in humans. tABI is based on the AE effect, an interaction between an ultrasound beam and tissue resistivity. The induced AE modulation is detected according to Ohm's law as a voltage across two or more recording electrodes. The AE signal Vᵢ^{AE} recorded by lead i at position \vec{r}(x,y,z) at ultrasound propagation time t is given by

$${V}_{i}^{AE} ( \vec{r} , t ) = - K {P}_{0} {ρ}_{0} \int \int \int ( {\tilde{J}}_{i}^{L} \cdot {J}^{I} ) b ( \vec{r} - {\vec{r}}^{′} ) a ( t - \frac{z}{c} ) d {\vec{r}}^{′3} , \qquad ( 8 )$$

where \tilde{J}ᵢ^{L}(\vec{r}) is the lead field, J^{L}(\vec{r}) is current density distribution, b(\vec{r}) is ultrasound beam pattern, and a(t) is ultrasound pulse waveform (see for the full derivation). Note this equation includes both the low frequency physiologic signal (such as EEG), as well as the high frequency AE modulation produced by the ultrasound beam. Thus, both signals can be captured on the same electrodes and separated by filters. The inventors have demonstrated feasibility of AE imaging in a variety of applications ranging from the live rabbit heart to most recently a human head phantom with embedded dipoles that produce EEG-like current sources. However, previous work has employed primarily single element focused ultrasound transducers or linear arrays at frequencies that do not readily penetrate the human skull (>1 MHz). This disclosure describes the performance of a novel 2D ultrasound array designed for 4D (volume+time) ABI with electronic beam-steering through the human skull. The inventors demonstrate that current sources within physiologic range can be detected through human skull at depths greater than 40 mm.

2.2. Methods

A. 2D Ultrasound Array for tABI in Humans

A novel handheld 2D ultrasound array with 126 elements (18×7) was designed specifically for 4D tABI (referred to as H235). The design was first modeled in FOCUS™ simulation software and then fabricated by Sonic Concepts™. The center frequency of 0.6 MHz facilitated delivery through human skull for tABI experiments. The elevation axis (y) had a radius of curvature of 35 mm. The acoustic pressure, bandwidth and beam pattern were measured and calibrated with an Onda hydrophone (HGL200) with and without placement of a human skull cap, which provided an estimate of attenuation due to bone.

B. Experimental Setup

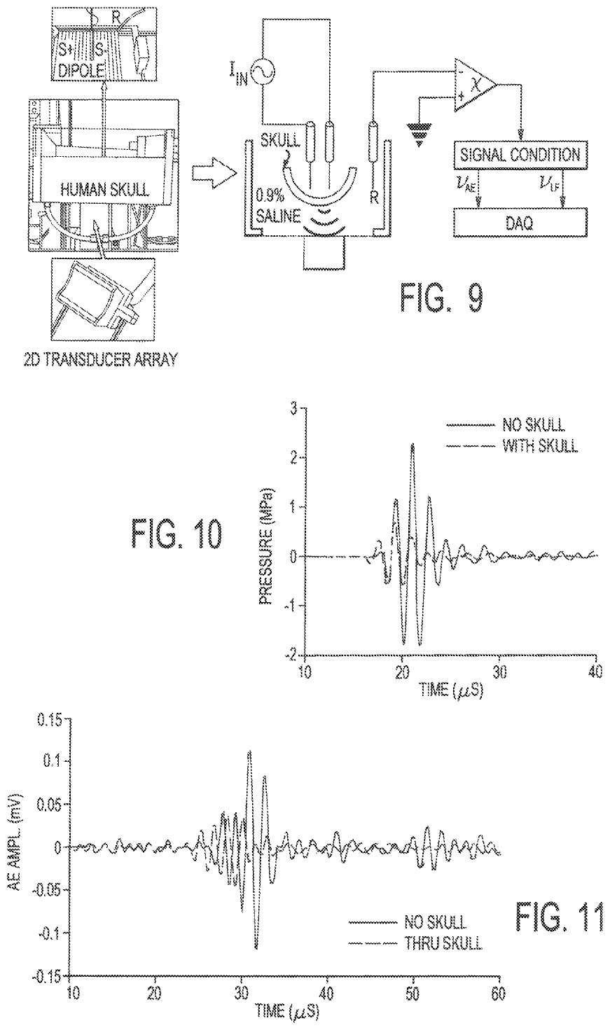

The experimental setup with human skull is depicted in FIG. 9. The skull cap was provided by the Will Body Program at the University of Arizona. The 2D ultrasound array was driven by the Verasonics® ultrasound platform (Vantage 64 LE™) to control acoustic pressure and electronically steer the ultrasound beam in 3D. The beamforming algorithm assumed the speed of sound of water as the medium. Two platinum stimulation electrodes were separated by 10 mm, immersed in 0.9% saline, and used to inject time-varying current into the medium. A copper wire electrode was placed in saline several centimeters from the stimulating wire for detecting the high frequency AE signal and low frequency current. A custom multichannel signal conditioning system was used to separate, filter, and amplify the high and low frequency signals.

C. Current Generation and Data Acquisition

An arbitrary function generator (Agilent 33220A) was used as a source for the current injection and a trigger for data acquisition (National Instruments PXI 1042). The timing between current injection and ultrasound pulsing was detailed in previous work. A 3-cycle 200-Hz current was injected into the medium. The high frequency AE signals were collected by the NI-PXI 5105 digitizer at a 20-MHz sampling rate, and the low frequency current signals were collected by the NI PXI 6289 DAQ card sampled at 20 kHz.

D. Signal and Image Processing

A band-pass filter (0.3-0.9 MHz pass band) was applied to each AE signal. Another band-pass filter (100-300 Hz) was applied along the physiological time axis for imaging. Each AE signal was also demodulated to produce magnitude AE images. The signal was further basebanded to produce color M-mode images with intensity and color indicating the strength and direction of the local current densities, respectively.

2.3. Results

A. Pressure Calibration for 2D Ultrasound Array

The experimental setup for pressure calibration is similar to that shown in FIG. 10. The H235 2D array was driven by the Verasonics ultrasonic system. The ultrasound beam passed through an acoustic window made of Mylar and into the surrounding medium. The ultrasound beam was focused on the tip of the hydrophone, which was located 35 mm above the transducer elements.

Driven by a 20V short pulse, the ultrasound transducer array yielded a 2.33 MPa positive peak pressure and a −1.78 MPa negative peak pressure without the skull cap (Mechanical Index=2.3). With the skull cap inserted (thickness=˜8 mm) between the ultrasound array and hydrophone, the positive and negative peak pressures were 0.68 and −0.53 MPa respectively (Mechanical Index=0.68). This corresponded with a decrease of 71% attenuation due to the skull.

B. Performance of tABI

To acquire baseline estimates of the sensitivity and resolution for the 2D array, as well as attenuation affects due to the skull, the inventors conducted two similar experiments; first without the skull, followed by with the skull inserted between the H235 and the dipole. In both scenarios, a time-varying dipole (3-cycle 200-Hz) was generated far away (>40 mm) from the bottom surface (Mylar or skull) by two platinum electrodes connected to the function waveform generator. AE signals were acquired at 2 kHz (every 0.5 milliseconds). Filtered AE signals (A lines) at the peak of the injected current are displayed in FIG. 11.

Without the skull cap, the peak-peak amplitude of the AE signal was 228 μV. With the skull cap, the AE peak-peak dropped by 70% to 69 μV, which was consistent with the drop in pressure estimated by the hydrophone.

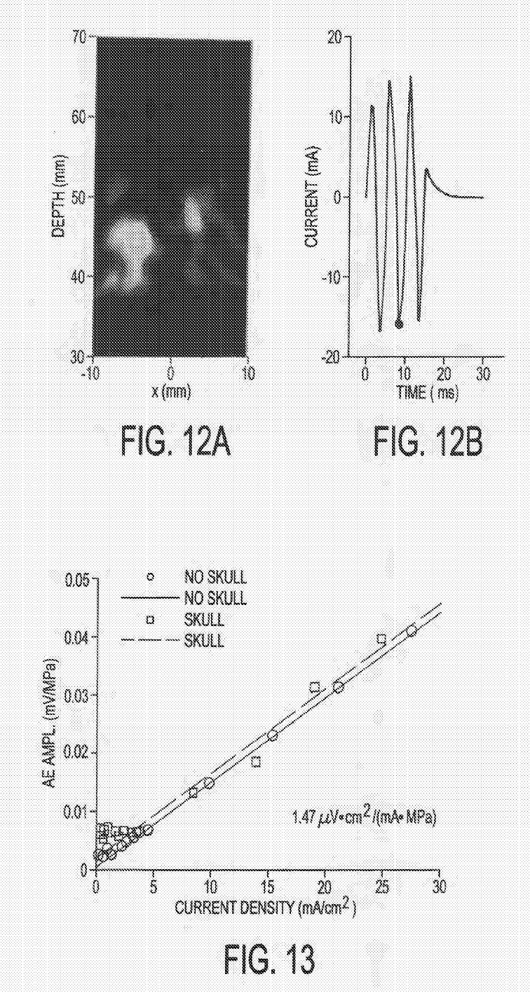

With the skull cap inserted, a B mode AE image was acquired by electronically steering the ultrasound beam along the lateral direction. FIG. 12A depicts one frame of a time-varying B mode movie representing the magnitude of the AE signal (at t=9 ms, per FIG. 12B). The strongest signal appears at the tip of the electrode (i.e., location of highest current density).

Because the current densities in the brain are typically less than 1 mA/cm², it is essential to estimate the detection threshold for these initial experiments. For the dipole setup in FIG. 9, the sensitivity of the 2D ultrasound array was determined by varying the current level while detecting the AE signal. Based on a linear fit, the sensitivity, when scaled for units of pressure, was similar: 1.47 μV·cm²/(mA·MPa) as displayed in FIG. 13.

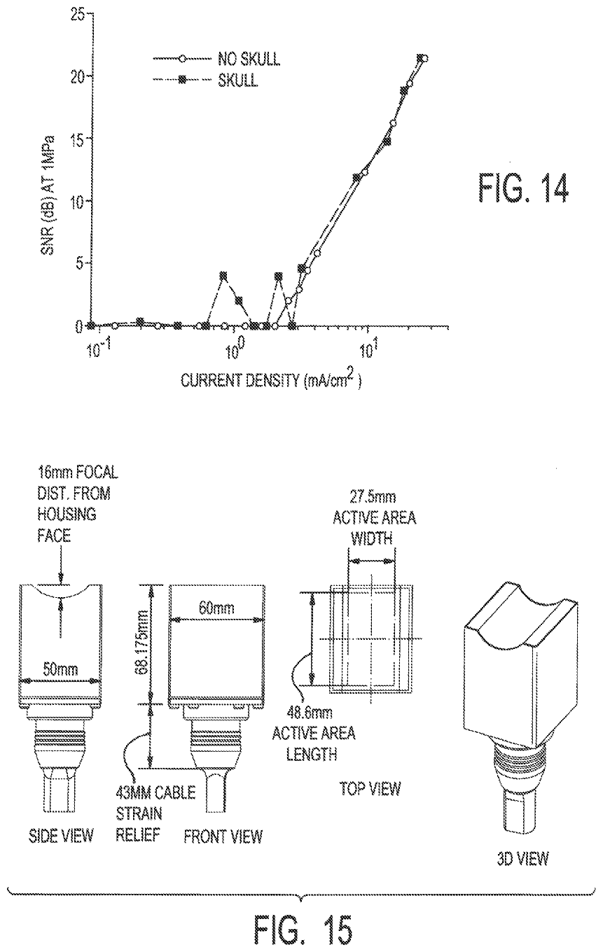

The signal-to-noise ratio (SNR) was evaluated at different current density levels at a peak pressure of 0.7 MPa, as indicated in FIG. 14. The noise equivalent current densities normalized to per MPa were 1.3 and 1.8 mA/cm² with and without skull, respectively.

2.4. Discussion and Conclusion

The inventors demonstrated for the first time transcranial ABI through a human skull using a custom 2D ultrasound array capable of real-time 4D tABI. Baseline estimates of the detection threshold through an adult human skull were 1.8 mA/cm². However, the inventors expect the sensitivity to dramatically improve after employing frequency encoded ultrasound excitation, which by-itself can improve SNR by more than 10 dB without sacrificing spatial resolution, ultrafast plane wave sequences, and/or increasing the # of trial averages. With further optimization, the detection limits should extend well beyond the range of physiologic neural currents (<1 mA/cm²). Although spatial resolution was 4 to 5 mm for this example, the inventors expect to be able to approach the diffraction limit (˜2 mm at 0.6 MHz) in other examples by employing a beamforming algorithm that considers the speed of sound of bone, as well as the surrounding medium/tissue. This will also improve the focusing capability of the 2D array and, thereby, improve sensitivity.

With further optimization and refinement of AE technology, tABI could evolve into a revolutionary tool for safe, real-time and high resolution electrical brain imaging in humans. Such a modality would have a profound impact on our understanding of the brain, human behavior, diagnosis and guiding treatment decisions for major neural disorders.

FIG. 15 is a diagram illustrating geometry of an exemplary transcranial transducer array. A two-dimensional wideband ultrasound transducer array with 126 elements (18×7) has been designed for 4D (volume+time) non-invasively mapping of electrical current in the brain through the skull. The transducer array may have a center frequency of substantially 0.3 MHz-5 MHz and >50% fractional bandwidth. More specifically, the center frequency and bandwidth may be designed to be, for example, 0.6 MHz and 0.41 MHz, respectively, for optimizing ultrasound penetration through bone and detecting electrical current at high spatial resolution. The aperture is rectangular in shape (49.00 mm×33.50 mm) with concave-cylindrical focus of 25 mm along the elevational direction. Element pitch is 2.7 mm wide and 4.18 mm high. The element kerf is 0.25 mm. The 2D design provides the capability of 4D electrical current mapping with electrical beamsteering without the need of physically moving the ultrasound probe. The design of this array was optimized for high spatial transcranial electrical brain imaging to better understand brain function, diagnose and guide-treatment for a variety of neurologic disorders. In addition to its application for acoustoelectric brain imaging (ABI), this probe has unique capabilities for 3D pulse echo ultrasound (tissue structure, motion, bone thickness) and transcranial doppler blood flow imaging. The handheld probe may have niche applications beyond the capabilities of existing ultrasound arrays designed for the human head therapy (e.g., high intensity focused ultrasound (HIFU) therapy) and imaging (transcranial doppler phased array). These techniques require a helmet array (e.g., for HIFU) or delivery of ultrasound through an acoustic window (typically the temporal window), which only gives access to a limited volume in the brain. The handheld device also interfaces with an open platform ultrasound delivery system for applications related to imaging, neuromodulation, and therapy. A handheld ultrasound transducer array for 3D or 4D transcranial ultrasound imaging, acoustoelectric imaging, and related modalities is therefore herein described.

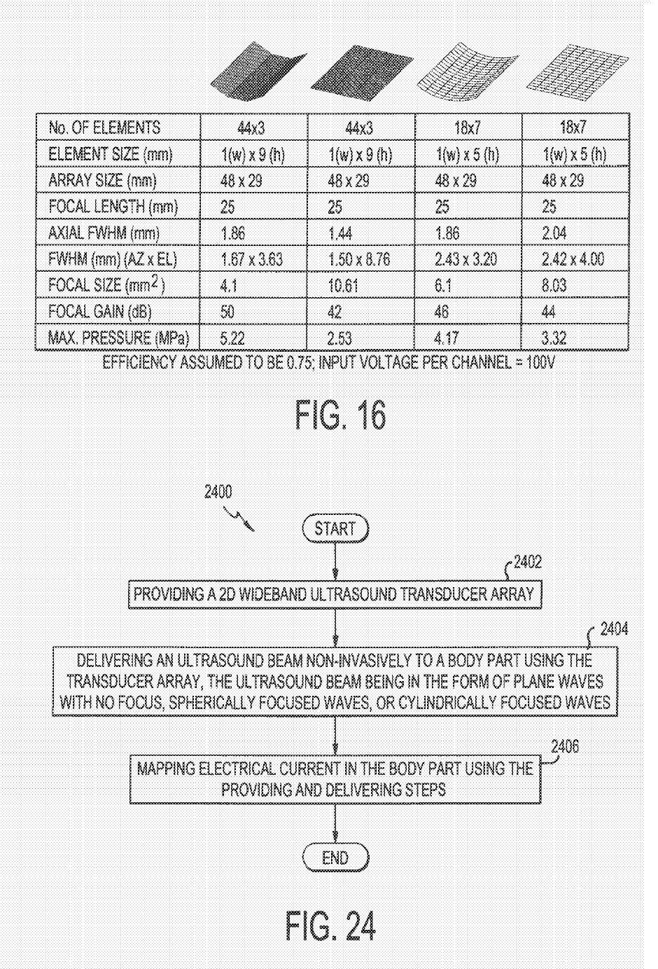

FIG. 16 is a diagram illustrating a comparison chart of parameters for various transcranial transducer arrays. Efficiency is assumed to be 0.75; and input voltage per channel=100 V.

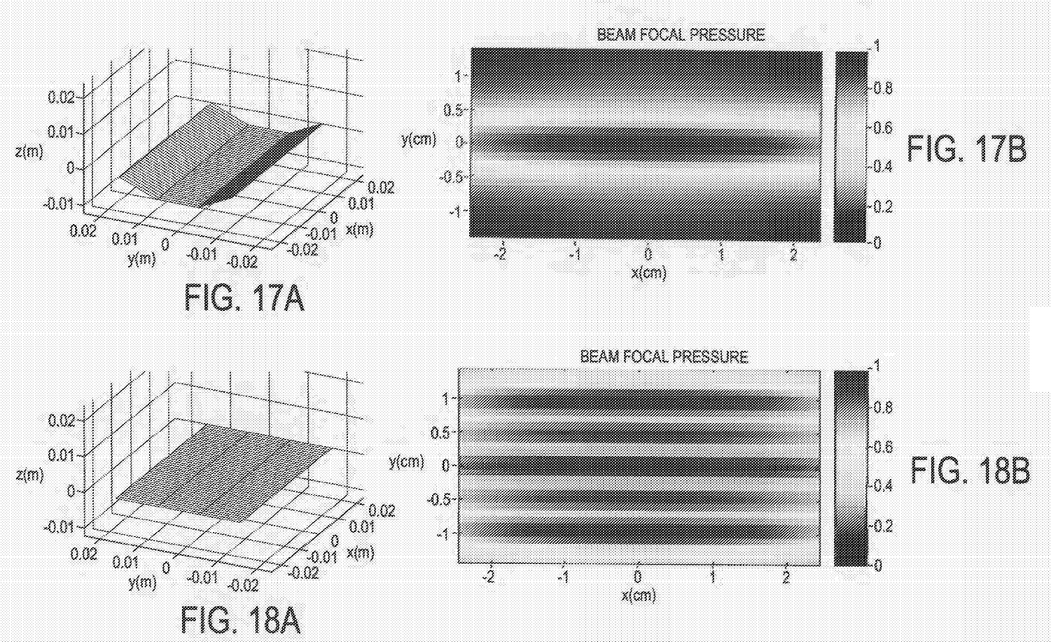

FIG. 17A is a diagram illustrating a 44×3 curved strip transcranial transducer array. FIG. 17B shows pressure at beam steered focuses. To compare pressure at desired focus and natural focus, acoustic beam was steered and resultant focal pressure was mapped to each position in x-y plane (z=5 cm).

FIG. 18A is a diagram illustrating a 44×3 flat strip transcranial transducer array. FIG. 18B shows pressure at beam steered focuses. To compare pressure at desired focus and natural focus, acoustic beam was steered and resultant focal pressure was mapped to each position in x-y plane (z=5 cm).

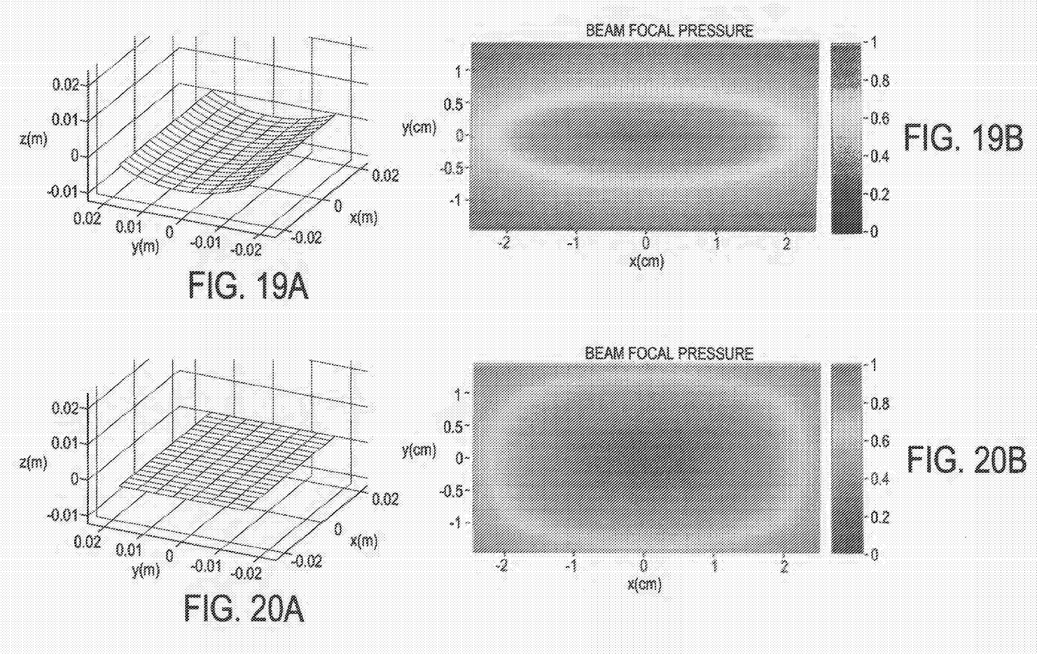

FIG. 19A is a diagram illustrating a 18×7 curved strip transcranial transducer array. FIG. 19B shows pressure at beam steered focuses. To compare pressure at desired focus and natural focus, acoustic beam was steered and resultant focal pressure was mapped to each position in x-y plane (z=5 cm).

FIG. 20A is a diagram illustrating a 18×7 flat strip transcranial transducer array. FIG. 20B shows pressure at beam steered focuses. To compare pressure at desired focus and natural focus, acoustic beam was steered and resultant focal pressure was mapped to each position in x-y plane (z=5 cm).

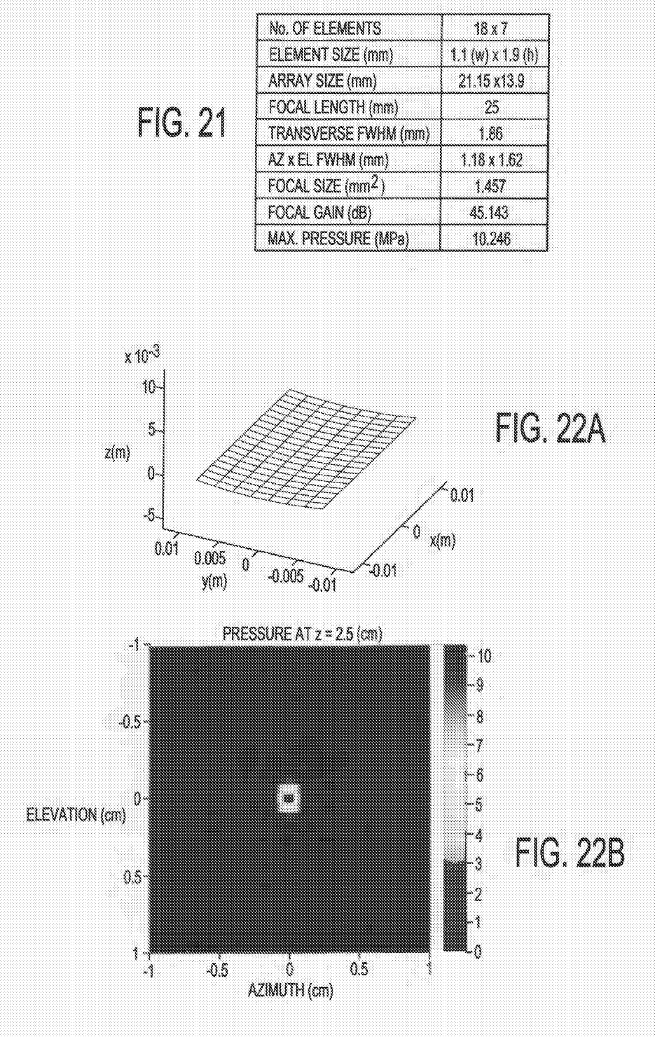

FIG. 21 is a diagram illustrating a chart of parameters for a transthoracic transducer array.

FIG. 22A is a diagram illustrating a 18×7 curved strip transthoracic transducer array. FIG. 22B shows a plot of pressure vs. elevation (depicting pressure at beam steered focuses).

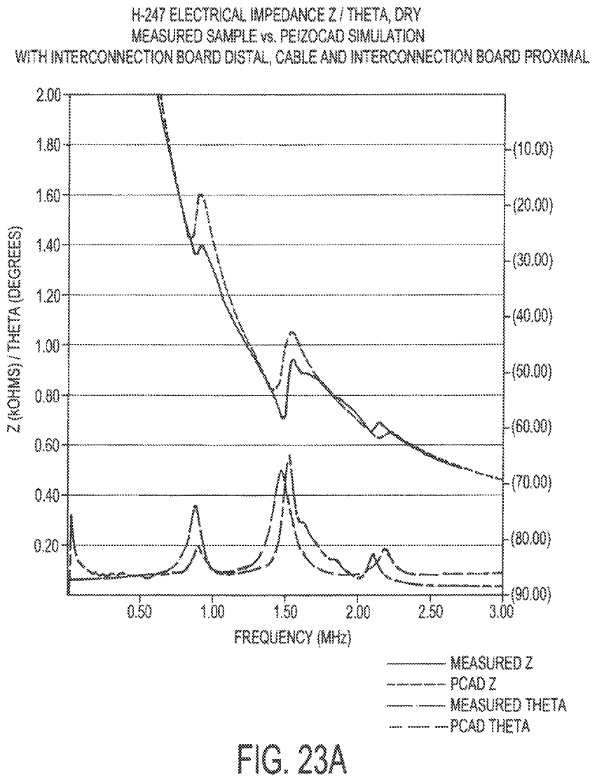

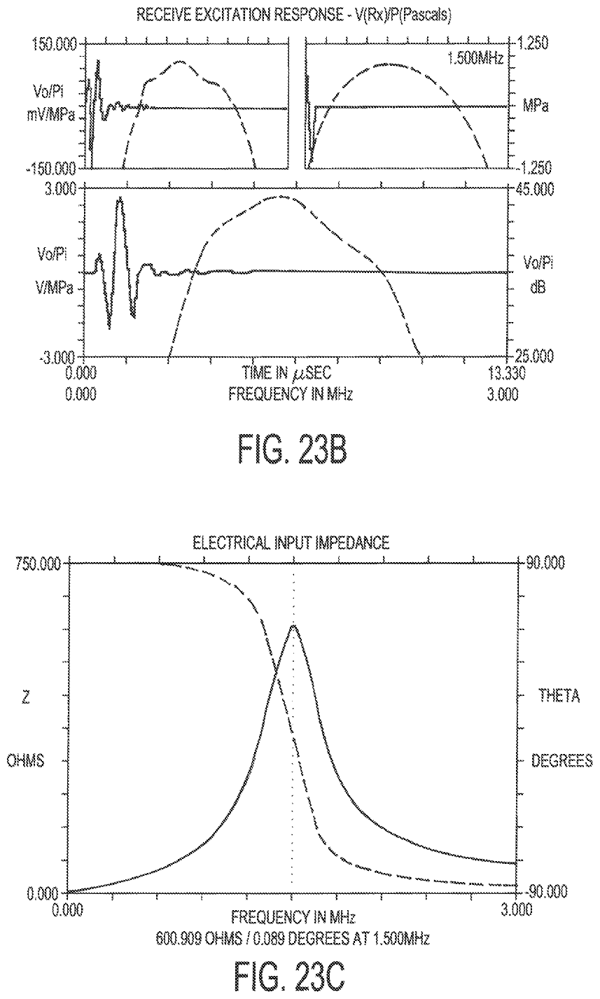

FIGS. 23A-23C are diagrams illustrating electrical impedance (FIG. 23A), received excitation response (FIG. 23B), and electrical input impedance (FIG. 23C), all vs. frequency. Measured and Simulated results for this 18×7 curved strip transthoracic transducer array design are illustrated in Table 1 below.

TABLE 1
Measured and Simulated results for one element: exemplary design
Center Frequency, Bandwidth, and Pulse Width

| | Center | | | |
|---|---|---|---|---|
| Level | Frequency | Bandwidth | Bandwidth | Pulse Width |
| (dB) | (MHz) | (MHz) | (% CF) | (μsec) |
| −6 | 1.398 | 1.070 | 76.54 | 0.803 |
| −20 | 1.491 | 1.800 | 120.73 | 1.680 |
| −40 | 1.540 | 2.472 | 160.48 | 3.516 |

Peak Amplitude = 8.51 dB re 1 V/MPa
Impulse = 16.61 dB re 1 V/MPa

Embodiments may provide for an understanding of human behavior, diagnosing and treating neurologic disease and brain injury. Existing techniques are limited by poor spatial resolution (e.g., EEG) or can only measure slow metabolic signals (e.g., fMRI, PET). There is an unmet need for a modality for non-invasive, real-time, and high resolution imaging of electrical brain activity.

Embodiments are directed to a method for 3D or 4D non-invasive imaging. FIG. 24 is a flowchart illustrating an embodiment of a method 2400 for 3D or 4D non-invasive imaging, in accordance with an embodiment. The method 2400 comprises: providing a 2D wideband ultrasound transducer array (block 2402); delivering an ultrasound beam non-invasively to a body part using the transducer array, the ultrasound beam being in the form of plane waves with no focus, spherically focused waves, or cylindrically focused waves (block 2404); and mapping electrical current in the body part using the providing and delivering steps (block 2406). Embodiments of the method 2400 are capable of 3D or 4D mapping of electrical current in, for example, the brain through the skull, or the heart. The transducer may have curvature in the lateral and/or elevational directions to enhance focusing.

In an embodiment, the step of mapping is performed with electrical beam steering without the need of physically moving the transducer array.

In an embodiment, the step of mapping uses an imaging technique selected from the group consisting of acoustoelectric imaging, 3D pulse echo ultrasound, doppler blood flow imaging, and a combination thereof.

In an embodiment, the step of mapping uses acoustoelectric imaging.

In an embodiment, the transducer array comprises a rectangular aperture.

In an embodiment, the transducer array allows for excitation pulses with linear or nonlinear coding schemes.

In an embodiment, the method further comprises operating the transducer array at a center frequency of substantially 0.3 MHz-5 MHz and >50% fractional bandwidth. The body part may comprise the brain.

In an embodiment, the method further comprises operating the transducer array at a center frequency of substantially 1 MHz-20 MHz and >50% fractional bandwidth. The body part may comprise the heart.

Embodiments are also directed to a 3D or 4D non-invasive imaging system. In an embodiment, the non-invasive imaging system comprises: a 2D wideband ultrasound transducer array that delivers an ultrasound beam non-invasively to a body part, the ultrasound beam being in the form of plane waves with no focus, spherically focused waves, or cylindrically focused waves; and a mapping system that maps electrical current in the body part using information obtained via the ultrasound beam delivered by the transducer array. Embodiments of the system are capable of 3D or 4D mapping of electrical current in, for example, the brain through the skull, or the heart. The transducer may have curvature in the lateral and/or elevational directions to enhance focusing.

In an embodiment, the mapping system uses electrical beam steering without the need to physically move the transducer array to map the electrical current in the body part.

In an embodiment, the mapping system uses an imaging technique selected from the group consisting of acoustoelectric imaging, 3D pulse echo ultrasound, doppler blood flow imaging, and a combination thereof, to map the electrical current in the body part.

In an embodiment, the mapping system uses acoustoelectric imaging to map the electrical current in the body part.

In an embodiment, the transducer array comprises a rectangular aperture.

In an embodiment, the transducer array allows for excitation pulses with linear or nonlinear coding schemes.

In an embodiment, the transducer array is configured to operate at a center frequency of substantially 0.3 MHz-5 MHz and >50% fractional bandwidth. The body part may comprise the brain.

In an embodiment, the transducer array is configured to operate at a center frequency of substantially 1 MHz-20 MHz and >50% fractional bandwidth. The body part may comprise the heart.

Although embodiments are described above with reference to systems and methods for 3D or 4D non-invasive imaging of the brain via the skull, the systems and methods may alternatively or additionally be applied to other parts of the body such as the heart via, for example, the chest/ribs (as briefly described above). Such alternatives are considered to be within the spirit and scope of the present invention, and may therefore utilize the advantages of the configurations and embodiments described above. The inventors have achieved overcoming limitations with standard electroanatomical mapping (EAM) for ablation therapy by developing 4D acoustoelectric cardiac imaging (ACI), a revolutionary new modality for real-time, volumetric imaging of current flow and cardiac potentials in the heart. ACI introduces a pulsed ultrasound beam to modulate local tissue resistivity. As ultrasound interacts with cardiac currents, a voltage modulation (“AE signal”) is generated at the ultrasound frequency and detected by a recording electrode. This AE signal is proportional to the local current density and spatially confined to the ultrasound focus. By rapidly sweeping the ultrasound beam while simultaneously detecting the AE modulations, 4D current density images are produced. ACI offers real-time capability and superior spatial resolution (0.2-2 mm) for mapping the cardiac activation wave and localizing arrhythmias. The inventors' preliminary studies indicate that ACI would offer the following benefits over conventional EAM for tracking arrhythmias during ablation therapy.

In addition, although embodiments are described above with reference to the number of elements in the arrays as being 44×3 or 18×7 for the flat or curved transcranial transducer array, or 18×7 for the curved transthoracic transducer array, other arrays with different number (or size) of elements, different aspect ratios, and/or different shapes may alternatively or additionally be employed in either the transcranial transducer array or transthoracic transducer array. Moreover, any or all of the other parameters in FIGS. 16 and 21 may alternatively be different than as described. Such alternatives are considered to be within the spirit and scope of the present invention, and may therefore utilize the advantages of the configurations and embodiments described above.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A method for 3D or 4D non-invasive imaging, the method comprising:
providing a 2D wideband ultrasound transducer array;
delivering an ultrasound beam non-invasively to a body part using the transducer array, the ultrasound beam being in the form of plane waves with no focus, spherically focused waves, or cylindrically focused waves; and
mapping electrical current in the body part using the providing and delivering steps.

2. The method of claim 1, wherein the step of mapping is performed with electrical beam steering without the need of physically moving the transducer array.

3. The method of claim 1, wherein the step of mapping uses an imaging technique selected from the group consisting of acoustoelectric imaging, 3D pulse echo ultrasound, doppler blood flow imaging, and a combination thereof.

4. The method of claim 1, wherein the step of mapping uses acoustoelectric imaging.

5. The method of claim 1, wherein the transducer array comprises a rectangular aperture.

6. The method of claim 1, wherein the transducer array allows for excitation pulses with linear or nonlinear coding schemes.

7. The method of claim 1, further comprising operating the transducer array at a center frequency of substantially 0.3 MHz-5 MHz and >50% fractional bandwidth.

8. The method of claim 7, wherein the body part comprises the brain.

9. The method of claim 1, further comprising operating the transducer array at a center frequency of substantially 1 MHz-20 MHz and >50% fractional bandwidth.

10. The method of claim 9, wherein the body part comprises the heart.

11. A 3D or 4D non-invasive imaging system comprising:
a 2D wideband ultrasound transducer array that delivers an ultrasound beam non-invasively to a body part, the ultrasound beam being in the form of plane waves with no focus, spherically focused waves, or cylindrically focused waves; and
a mapping system that maps electrical current in the body part using information obtained via the ultrasound beam delivered by the transducer array.

12. The non-invasive imaging system of claim 11, wherein the mapping system uses electrical beam steering without the need to physically move the transducer array to map the electrical current in the body part.

13. The non-invasive imaging system of claim 11, wherein the mapping system uses an imaging technique selected from the group consisting of acoustoelectric imaging, 3D pulse echo ultrasound, doppler blood flow imaging, and a combination thereof, to map the electrical current in the body part.

14. The non-invasive imaging system of claim 11, wherein the mapping system uses acoustoelectric imaging to map the electrical current in the body part.

15. The non-invasive imaging system of claim 11, wherein the transducer array comprises a rectangular aperture.

16. The non-invasive imaging system of claim 11, wherein the transducer array allows for excitation pulses with linear or nonlinear coding schemes.

17. The non-invasive imaging system of claim 11, wherein the transducer array is configured to operate at a center frequency of substantially 0.3 MHz-5 MHz and >50% fractional bandwidth.

18. The non-invasive imaging system of claim 17, wherein the body part comprises the brain.

19. The non-invasive imaging system of claim 11, wherein the transducer array is configured to operate at a center frequency of substantially 1 MHz-20 MHz and >50% fractional bandwidth.

20. The non-invasive imaging system of claim 19, wherein the body part comprises the heart.
